(19) United States
(12) Patent Application Publication
(10) Pub. No.: US 2026/0369423 A1
(43) Pub. Date: Nov. 19, 2026

(54) ASSESSING CARDIOVASCULAR DISEASES RISK USING TIME-SERIES RETINAL SCANS AND LONGITUDINAL DATA

(71) Applicant: Optain Health, Inc., Charlotte, NC (US)

(72) Inventors: Zongyuan Ge, Melbourne (AU); Mingguang He, Hong Kong (CN); Zhihong Lin, Melbourne (AU); Wei Meng, Melbourne (AU); Jason Sun, Melbourne (AU)

(73) Assignee: Optain Health, Inc., Charlotte, NC (US)

(21) Appl. No.: 19/730,613

(22) Filed: Jul. 2, 2026

Related U.S. Application Data
(63) Continuation of application No. PCT/US2025/010058, filed on Jan. 2, 2025.
(60) Provisional application No. 63/618,099, filed on Jan. 5, 2024.

(51) Int. Cl. G16H 50/30

(52) U.S. Cl. CPC G16H 50/30

(57) ABSTRACT

Predicting cardiovascular diseases (CVD) may be preceded by subtle changes in various health indicators that may make early predictions useful for timely intervention. The present disclosure relates to prediction of one or more diseases, particularly CVD, for a given subject by leveraging one or more machine-learning models based on longitudinal multimodal data. The techniques, as disclosed herein, may utilize the one or more machine-learning models including one or more feature generators, a temporal aggregator and a prediction model to process the longitudinal multimodal data, generating relevant features, a temporal feature vector and one or more metrics corresponding to one or more diseases associated with the subject. The generated metrics based on the current time and previous time points may be analyzed to assess whether the subject currently has or is at risk of developing one or more diseases.

### CROSS-REFERENCE TO RELATED APPLICATIONS

This application is a continuation of International Patent Application No. PCT/US2025/010058, filed on Jan. 2, 2025, which claims the priority to and the benefit of U.S. Provisional Application No. 63/618,099, filed on Jan. 5, 2024, entitled “System and Method for Assessing Cardiovascular Disease Risk using Time-series Retina Images and Longitudinal Data”. The entire disclosures of the aforementioned applications are incorporated by reference herein in their entireties for all purposes.

### BACKGROUND

Cardiovascular disease (CVD) remains a global health challenge that affects millions of people worldwide. CVD includes conditions such as coronary heart disease, stroke, and hypertension that may lead to severe health complications and death. Therefore, early detection of CVD may assist in improving health status of a subject, yet it remains challenging due to the complexity of the vascular system. Unlike many diseases that present clear and noticeable symptoms, cardiovascular issues often develop silently with the vascular network hidden beneath the skin, making it difficult to identify early warning signs or subtle changes that may indicate the onset of cardiovascular problems. Traditional diagnostic techniques such as angiography, while providing detailed images of blood vessels, are invasive and often come with risks and/or discomfort. Moreover, such methods may only offer a snapshot of the vascular system at a specific point in time, limiting their ability to track a progression of the disease or detect subtle early changes.

Additionally, commonly used diagnostic tools such as blood pressure readings, cholesterol levels, and electrocardiograms (ECGs) provide valuable data but may lack to offer a holistic view of a subject's cardiovascular health. Results from such diagnostic tools are typically focused on isolated aspects of cardiovascular function and may not capture dynamic changes over time. These diagnostic tools may also overlook early subtle changes that contribute to the risk prediction of the future CVD events. One promising development is the use of retinal imaging e.g., fundus examinations, where retinal scans reveal the microvascular network of the eye. Retinal imaging may provide insights into the condition of the microvasculature with changes in the retinal blood vessels often reflecting broader systemic vascular health. Such changes may be indicative of underlying cardiovascular diseases including hypertension and diabetes, which may make retinal scans a diagnosing tool for early CVD detection.

However, retinal scans may not be sufficient on their own for a complete CVD diagnosis. The retinal vasculature may also show signs of degeneration due to natural aging processes that may confound an interpretation of retinal scans when considering them in isolation. For example, age-related macular degeneration or other age-related retinal changes may mimic the effects of vascular diseases leading to misinterpretation.

### SUMMARY

Some aspects of the present disclosure relate to techniques for predicting cardiovascular disease (CVD) risk based on time-series retinal scans and longitudinal data of a subject. The time-series retinal scans may correspond to retinal scans obtained over a set of time points, each potentially involving one or more retinal modalities. The longitudinal data may refer to non-retinal data collected over the same set of time points, which together with the retinal scans may form multimodal data over the set of time points (interchangeably used herein with longitudinal multimodal data). The disclosed techniques may include accessing the multimodal data of the subject that comprises a retinal scan and non-retinal data for each time point of the set of time points. The retinal scan associated with the one or more retinal modalities may include, but are not limited to, one or more of: fundus imaging, optical coherence tomography (OCT), fluorescence angiography (FA), functional magnetic resonance imaging (fMRI), ocular ultrasound imaging, multispectral imaging (MSI), scanning laser ophthalmoscopy (SLO) and ocular radiography.

The longitudinal data (interchangeably used herein with non-retinal data) of the subject may be obtained and/or derived from various sources, including but not limited to electronic health records, medical-provider observations, and/or input from the subject. The non-retinal data may include, but is not limited to, one or more of: demographic data (e.g., age, gender), anthropometric data (e.g., weight, height, body mass index), comorbidities data (e.g., diabetic status, hypertension), medical imaging data (e.g., MRI, X-ray of any body part apart from the retina), or lifestyle indicators (e.g., smoking status, alcohol consumption, physical activity level) corresponding to each time point of the set of time points.

A set of feature vectors associated with the multimodal data over the set of time points may be generated using one or more machine-learning models. Each feature vector of the set of feature vectors may correspond to a particular point in time. In some instances, each feature vector of the set of feature vectors further includes a first embedding vector and a second embedding vector. The first embedding vector (for example) associated with a set of image-based modalities may be generated via a first machine-learning model of the one or more machine-learning models. Similarly, the second embedding vector (for example) associated with a set of text-based modalities may be generated via a second machine-learning model of the one or more machine-learning models.

The image-based modalities may comprise images from one or more retinal modalities or the non-retinal data. For example, the set of image-based modalities may include images from one or more retinal modalities (e.g., fundus or OCT images) and non-retinal data (e.g., X-ray and MRI of any body part apart from the retina). Similarly, the set of text-based modalities may include textual data associated with the non-retinal data e.g., demographic data, medical notes etc. The first and the second machine-learning models may be (for example) neural networks configured to generate and process embedding vectors from the set of image-based modalities and the set of text-based modalities. In some instances, the first and the second machine-learning models may be a unified multimodal model configured to generate and process embedding vectors jointly from both the set of image-based modalities and the set of text-based modalities.

The set of feature vectors associated with the set of time points may be aggregated to generate a temporal feature vector. A third machine-learning model of the one or more machine-learning models may be leveraged to generate the temporal feature vector, where the model may include (for example) a sequence-based model configured to capture changes over the set of time points. In some instances, when the first embedding vector and the second embedding vector are generated separately, these vectors may be first aggregated to form a multimodal fusion vector and the multimodal fusion vectors across all time points may be aggregated again to generate a temporal feature vector.

A disease-prediction metric may be generated by processing the temporal feature vector using a prediction model of the one or more machine-learning models. In one aspect, the prediction model may be a single model that comprises a deep neural network to generate the disease-prediction metric. In another aspect, the prediction model may be a set of sub-models including, but not limited to: a Cox model (predicting disease progression or mortality risk), an incident model (predicting the likelihood of disease onset), an aging model (predicting age-related changes in health) and a patient information model (predicting risk based on medical history, lifestyle, and genetics). Predictions from the set of sub-models may be integrated using an ensemble model that may combine weighted predictions of each sub-model of the set of sub-models to generate the disease-prediction metric. The one or more machine-learning models including the first, second and the third machine-learning model, and the prediction model may be trained jointly in an end-to-end manner as a single machine-learning model. Additionally, the one or more machine-learning models may be trained using a loss function that may predict whether the set of feature vectors representing retinal degradation corresponds to aging or a disease incident. The predicted metric representing cardiovascular health status of the subject may be displayed in a graphical user interface (GUI).

In some aspects, an output result corresponding to the disease-prediction metric may be generated using the prediction model. The output result corresponding to the disease-prediction metric may include (for example) a risk score, a probability, or a predicted change. For example, the output result may represent and/or include (for example) a predicted risk of the subject experiencing one or more specific cardiovascular disease (e.g., a heart attack or stroke) within a defined time period. The risk score may represent (for example) a predicted probability that the subject currently has a cardiovascular disease (e.g., generally or of a specific type), a predicted severity of any cardiovascular disease of the subject, a predicted probability that the subject will have a cardiovascular disease (e.g., of any type or of a specific type) within a defined time period, and/or a predicted severity of a cardiovascular disease of the subject at a given future time point. The output result may further present and/or include prediction of one or more other diseases including (for example) one or more of: diabetic retinopathy, age-related macular degeneration, glaucoma, hypertensive retinopathy, stroke, multiple sclerosis, Alzheimer's disease, Parkinson's disease, chronic kidney disease, retinal vein occlusion, macular edema, systemic inflammatory diseases including lupus and rheumatoid arthritis and/or ocular or metastatic cancers.

In some aspects, a system is provided that includes one or more data processors and a non-transitory computer readable storage medium containing instructions which, when executed on the one or more data processors, cause the one or more data processors to perform part or all of one or more methods disclosed herein.

In some aspects, a computer-program product tangibly embodied in a non-transitory machine-readable storage medium, including instructions configured to cause one or more data processors to perform part or all of one or more methods or processes disclosed herein.

In some aspects, a system is provided that includes one or more means to perform part or all of one or more methods or processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by aspects and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

Various embodiments are described hereinafter with reference to the figures. It should be noted that the figures are not drawn to scale and that the elements of similar structures or functions are represented by like reference numerals throughout the figures. It should also be noted that the figures are only intended to facilitate the description of the embodiments. They are not intended as an exhaustive description of the disclosure or as a limitation on the scope of the disclosure.

FIG. 1 illustrates an example overview predicting one or more diseases particularly cardiovascular diseases (CVD) based on one or more retinal modalities and non-retinal data of a subject in accordance with some aspects of the present disclosure.

FIG. 2 depicts an example timeline illustrating longitudinal multimodal data collected at multiple time points in accordance with some aspects of the present disclosure.

FIG. 3 illustrates an example overview of a computing system of the FIG. 1 to predict the one or more diseases based on the longitudinal multimodal data of the subject in accordance with some aspects of the present disclosure.

FIG. 4 illustrates a detailed block diagram from the FIG. 3, predicting one or more diseases based on the longitudinal multimodal data in accordance with some aspects of the present disclosure.

FIG. 5 illustrates a block diagram depicting a process of aggregating features from the longitudinal multimodal data into a multimodal feature vector in accordance with some aspects of the present disclosure.

FIG. 6 illustrates an example block diagram depicting a process of aggregating one or more multimodal feature vectors into a temporal feature vector to make predictions in accordance with some aspects of the present disclosure.

FIG. 7 illustrates an exemplary training of one or more machine-learning (ML) models, utilizing a loss function in accordance with some aspects of the present disclosure.

FIG. 8 illustrates prediction of the one or more diseases using a set of sub-models to separately process one or more factors associated with the temporal feature vector in accordance with some aspects of the present disclosure.

FIG. 9A shows an illustrative example of a first graphical user interface (GUI) representing a dashboard for tracking and accessing the one or more diseases in accordance with some aspects of the present disclosure.

FIG. 9B shows an illustrative example of a second GUI representing a report section based on the longitudinal multimodal data in accordance with some aspects of the present disclosure.

FIG. 10 illustrates an exemplary workflow to predict the one or more diseases using the longitudinal multimodal data in accordance with some aspects of the present disclosure.

FIG. 11 illustrates an exemplary block diagram of a computing system in which various aspects of the disclosed techniques may be executed.

### DETAILED DESCRIPTION

The present disclosure relates to techniques for analyzing longitudinal multimodal data (e.g., time-series retinal scans and snapshot data) to predict risk assessment for a subject. The predicted risk assessment associated with the subject may include, but is not limited to, a presence (or absence of a cardiovascular disease (CVD)), a CVD risk score (or a probability of developing the CVD at one or more time points), and/or predicted changes in CVD trends. For example, the prediction may estimate a likelihood of the subject experiencing specific cardiovascular events (e.g., heart attack, stroke) within a defined set of time points, the probability of current CVD presence, severity of the current CVD, or a future risk of developing CVD. The techniques, as disclosed herein, may further include prediction of one or more other diseases, in addition to CVD, including diabetic retinopathy, age-related macular degeneration, glaucoma, hypertensive retinopathy, stroke, multiple sclerosis, Alzheimer's disease, Parkinson's disease, chronic kidney disease, retinal vein occlusion, macular edema, systemic inflammatory diseases (e.g., lupus, rheumatoid arthritis), and ocular or metastatic cancers.

Assessing the risk of developing CVD and determining the progression of the one or more diseases may be complicated, in part, due to the variability in the appearance of the vasculature across subjects and across time within a subject. For instance, retinal blood vessels that show moderate tortuosity, dilation, and irregularities may be a normal sign of aging. However, in a younger subject, the same signs may indicate a rare vascular disease, such as Coates' disease. The disclosed techniques may include accessing multimodal data (e.g., retinal scan and snapshot data) associated with the subject to predict the risk of CVD incidents occurring in the subject.

The retinal scan for each time point may be associated with (or derived from) one or more retinal modalities including fundus imaging, optical coherence tomography (OCT), fluorescence angiography (FA), functional magnetic resonance imaging (fMRI), ocular ultrasound imaging, multispectral imaging (MSI), scanning laser ophthalmoscopy (SLO) and optical radiology. Similarly, the snapshot data corresponding to each time point may be derived from non-retinal data associated with the subject. The non-retinal data may include, but is not limited to, demographic data (e.g., age, gender), anthropometric data (e.g., weight, height, body mass index), comorbidities data (e.g., diabetic status, hypertension), medical imaging data (e.g., MRI, X-ray results), or lifestyle indicators data (e.g., smoking status, alcohol consumption, physical activity level). The snapshot data may be collected from various sources including an electronic health record, medical-provider observation, input from the subject, and the like.

The disclosed techniques may further access longitudinal multimodal data stored in a database, where the longitudinal multimodal data may include retinal scans and snapshot data from current and previous time points. The data storage in the database may support continuous monitoring of the health status of the subject and may enable health-care providers to make informed decisions about the CVD risks. The predictions generated by the disclosed techniques may also potentially be stored and/or further analyzed in the database. The longitudinal multimodal data may be (for example) analyzed and processed on a computing system. The computing device may be a device operated by the subject, a medical provider associated with treating the subject, or an entity facilitating medical monitoring or treatment for the subject. The longitudinal multimodal data may be communicated to the computing system over a network. The network may include communication protocols such as wired connections (e.g., Ethernet, USB, or fiber optics) or wireless connections (e.g., Wi-Fi, Bluetooth, or cellular networks).

The computing system may be running a risk assessment module that may further comprise advanced pre-processing modules and one or more machine-learning (ML) models for subsequent processing. The pre-processing modules may standardize and prepare the longitudinal multimodal data for proper formatting prior to subsequent analysis. In some aspects, the pre-processing may include segmenting the longitudinal multimodal data into different modalities for subsequent processing (e.g., image-based modalities and text-based modalities). The image-based modalities may comprise retinal scans from one or more retinal modalities and medical imaging data from the non-retinal data. Similarly, the text-based modalities may comprise textual data from the non-retinal data.

Following the pre-processing, the risk assessment module may use the one or more ML models for generating features, aggregating the generated features and generating a prediction based on the aggregated features. For feature generation, a first ML model (also termed as an image feature generator) and a second ML model (also termed as a text feature generator) may generate (for example) a first embedding vector and a second embedding vector based on image-based modalities and text-based modalities at a specific time point. The image feature generator and the text feature generator may include neural networks that may be configured to generate and process features from the associated modalities. The first embedding vector may correspond to an image embedding vector and the second embedding vector may correspond to a text embedding vector. In some instances, the image feature generator and the text feature generator may work as a single feature generator comprising a multimodal model configured to generate and process features from both the image-based modalities and text-based modalities.

The first embedding vector and second embedding vector may be aggregated using a multimodal fusion module to generate a multimodal fusion vector. The multimodal fusion module may generate a multimodal fusion vector. The fusion process may enable the risk assessment, capturing dynamic changes and temporal relationships across different modalities over time. Resultant multimodal fusion vector over different time points may then be aggregated using a temporal module. The temporal module may use techniques (for example) normalization, concatenation, weighted averaging, or RNNs/LSTMs to capture temporal relationships across the multiple time points and generate a temporal feature vector. In the temporal module, the multimodal fusion vector over different time points (herein after, referred to as a set of feature vectors) may be normalized. Based on such normalized vectors, a cumulative vector and a differential vector may be computed that may capture the global trends and may highlight local changes between consecutive time points. The cumulative vector and differential vectors may then be concatenated to form the temporal feature vector. The temporal module may use models like RNNs or LSTMs to process the aggregated data, where the RNN/LSTM may store the cumulative and the differential vectors from previous time points, allowing it to incorporate temporal dependencies. Such models may sequentially update the temporal feature vector, capturing long-term trends and short-term fluctuations. The integration of such temporal information including the retinal scans and longitudinal data may provide relevant information about the aging progress and change in vessel health status of the subject, which may be beneficial for prediction of CVD or other one or more diseases. The temporal feature vector may then be fed into a prediction model of the one or more ML models to generate a disease-prediction metric.

In one aspect, the prediction model may be a single model that comprises a deep neural network to generate the disease-prediction matric. In another aspect, the prediction model may be a set of sub-models including, but are not limited to, a Cox model, an incident model, an aging model or a patient information model. Each sub-model within the set of sub-models may analyze distinct factors including but not limited to disease history, clinical data, aging progression, and patient-specific characteristics. Predictions from the set of sub-models may be integrated using an ensemble model that may combine the weighted predictions of each sub-model of the set of sub-models to generate the disease-prediction metric.

The one or more ML models including the one or more feature generator, the temporal module and the prediction model may be trained jointly in an end-to-end manner as a single machine-learning model. The one or more ML models may be trained using a loss function that may predict whether the set of feature vectors representing retinal degradation corresponds to aging or a disease incident. Discrepancies between predicted results (e.g., CVD risk scores, other one or more diseases or the like) and true outcomes may guide model adjustments through backpropagation. A feature ranking module may use the loss information to prioritize among features that significantly impact the accuracy of the one or more models. The predicted metric representing the health status of the subject may then be displayed in a graphical user interface (GUI).

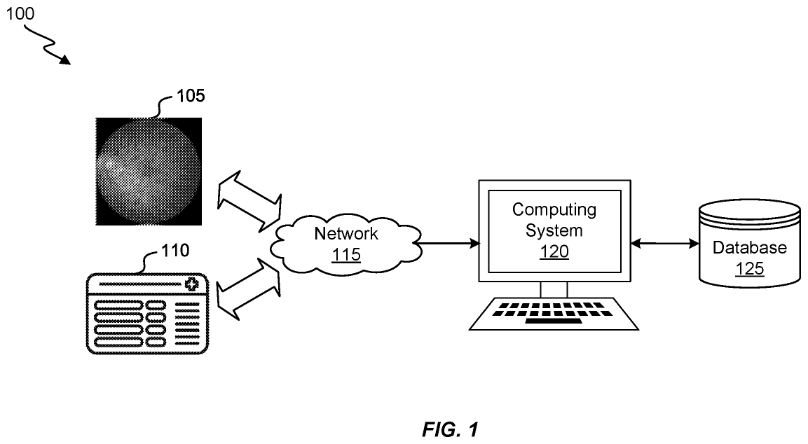

FIG. 1 illustrates an example overview 100 predicting one or more diseases particularly cardiovascular diseases (CVD) based on one or more retinal modalities and non-retinal data of a subject in accordance with some aspects of the present disclosure. Exemplary overview 100 comprises a retinal scan 105 (corresponding to a retinal modality of the one or more retinal modalities), snapshot data 110 (corresponding to the non-retinal data or longitudinal multimodal data at a specific time point), a network 115, a computing system 120 and a database 125. The retinal scan 105 and the snapshot data 110 of the subject may be transmitted to the computing system 120 via the network 115.

The computing system 120 may be operated by the subject, a clinician or a medical provider associated with treating the subject, or an entity facilitating medical monitoring or treatment for the subject. The computing system 120 may include a mobile device (e.g., a smartphone), personal digital/data assistants (PDA), a tablet, a laptop, a desktop computer, a computer server, and the like. The retinal scan 105 and snapshot data 110, which may be from a clinician, a camera capturing retinal images, an electronic health record, a medical-provider observation, and/or subject input, may be communicated to the computing system 120 over the network 115. The network 115 may be a communication infrastructure that may facilitate the transfer of multimodal data (e.g., the retinal scan 105 and the snapshot data 110) between the clinician's or medical device's interface and the computing system 120. The network 115 may include communication protocols such as wired connections (e.g., Ethernet, USB, or fiber optics) or wireless connections (e.g., Wi-Fi, Bluetooth, or cellular networks).

The computing system 120 may execute one or more ML models to analyze and process the multimodal data to predict one or more diseases. The results generated by the computing system 120 may be potentially stored or further analyzed in the database 125. Additionally, longitudinal multimodal data such as retinal scans and snapshot data from previous time points may also be stored in the database 125, which may enable the computing system 120 to track changes over time and make more accurate predictions based on historical information. Such data storage may allow for continuous monitoring of the health status of the subject, facilitating long-term analysis and supporting more informed decision-making by the healthcare providers.

The multimodal data may be stored in a memory of the computing system 120 or the database 125, which may be built within the computing system 120 or on a cloud-based storage system. While the multimodal data is illustrated as being stored at a single location, it should be appreciated that this is not intended to be limiting, the multimodal data may be stored in multiple memories or locations. The computing system 120 may be where the computations are carried out including the disclosed one or more ML models that may assist in predicting risk associated with CVD or one or more other diseases.

For instance, the prediction of CVD may estimate the likelihood of a subject experiencing specific cardiovascular events (e.g., heart attack, stroke) within a defined set of time points, the probability of current CVD presence, the severity of existing CVD, or a future risk of developing CVD. Further, conditions including atherosclerosis, coronary artery disease, heart failure, stroke, hypertension, arrhythmias and elevated cholesterol levels may be considered as early indicators or risk factors for CVD. Exemplary overview 100 may further include prediction of the one or more other diseases, in addition to CVD, including diabetic retinopathy, age-related macular degeneration, glaucoma, hypertensive retinopathy, stroke, multiple sclerosis, Alzheimer's disease, Parkinson's disease, chronic kidney disease, retinal vein occlusion, macular edema, systemic inflammatory diseases (e.g., lupus, rheumatoid arthritis), and ocular or metastatic cancers.

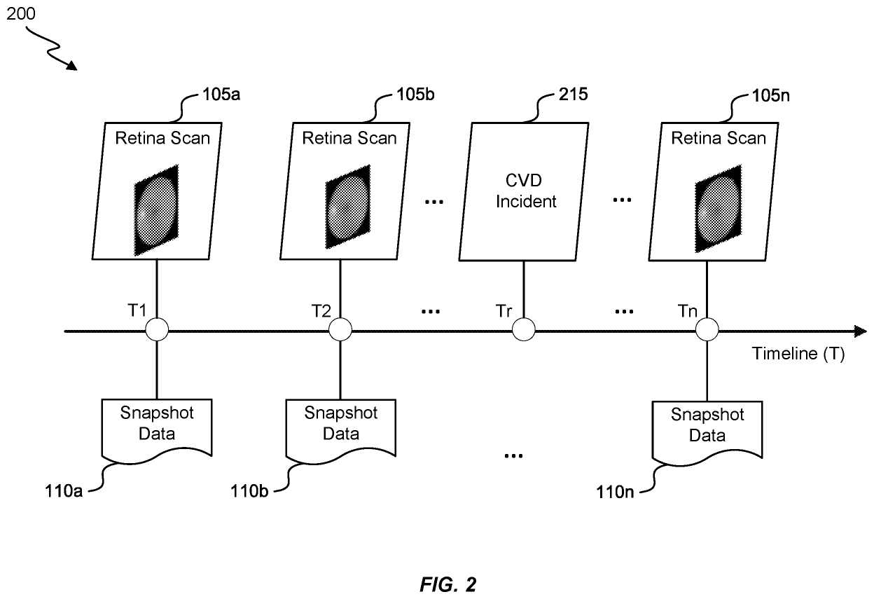

FIG. 2 depicts an exemplary timeline 200 illustrating longitudinal multimodal data collected at multiple time points in accordance with some aspects of the present disclosure. Exemplary timeline 200 captures the multimodal data of the subject and organizes the multimodal data along a set of time points denoted as T1, T2, . . . , Tn, where T1, T2, . . . may represent earlier time points corresponding to the health status of the subject and Tn may represent the most recent or current time point. The set of time points (T1, T2, . . . , Tn) may refer to specific moments or intervals in time at which the multimodal data may be collected and/or analyzed. Exemplary timeline 200 maps retinal scans 105a, 105b, . . . , 105n (which may be collectively referred as retinal scans 105 and may be individually referred as retinal scan 105) and snapshot data 110a, 110b, . . . , 110n (which may be collectively referred as snapshot data 110 and may be individually referred as snapshot data 110), each corresponding to time points T1, T2, . . . , Tn of the set of time points. Thus, the multimodal data over multiple time points (e.g., from T1 to Tn) may form a record of the health status of the subject. Such time-series multimodal data may, hereafter, be termed as the longitudinal multimodal data or the multimodal data over a set of time points.

The multimodal data corresponding to an associated time point of the set of time points may not be limited to scheduled checkup or test, the multimodal data may be gathered continuously as part of the subject ongoing health monitoring. Retinal scans 105 may be derived from one or more retinal modalities that may include a variety of imaging techniques to capture detailed information about the retina and its associated structures. One such technique (for example) fundus imaging may provide high-resolution photographs of the retina that may allow for the detection of abnormalities such as diabetic retinopathy or retinal artery occlusions. In contrast, optical coherence tomography (OCT) utilizes light waves to create cross-sectional images of the retina that may lead to identification of retinal conditions including macular degeneration, glaucoma, and diabetic macular edema. Moving on, fluorescence angiography (FA) may involve injecting a dye into the bloodstream to capture real-time images of blood vessels in the retina. Functional magnetic resonance imaging (fMRI), although primarily used for studying brain activity, may also be applied to observe retinal blood flow and oxygenation changes particularly in studies related to retinal ischemia or optic nerve diseases.

While not typically used for retinal imaging, ocular ultrasound imaging may still assist in evaluating the eye's internal structures to diagnose conditions such as retinal detachment or ocular tumors. Moreover, multispectral imaging (MSI) may capture images across various wavelengths of light that may then offer a deeper understanding of tissue characteristics and abnormalities, which may enhance the detection of early retinal pathologies. Similarly, scanning laser ophthalmoscopy (SLO) uses laser light to scan the retina and produce detailed images that may provide high-resolution insights into retinal layers and blood vessels essential for diagnosing glaucoma and other retina-related diseases. Such anatomical features from imaging techniques may reflect systemic health conditions linked to CVD or the one or more other diseases.

Similarly, the snapshot data 110 for each time point may be derived from non-retinal data associated with the subject. The non-retinal data may include demographic data (e.g., age, gender), anthropometric data (e.g., weight, height, body mass index), comorbidities data (e.g., diabetic status, hypertension), medical imaging data (e.g., MRI, X-ray results), or lifestyle indicators data (e.g., smoking status, alcohol consumption, physical activity level). The snapshot data 110 may be aggregated from diverse sources, such as clinical laboratory diagnostics, self-reported health assessments, and electronic medical records (EMRs).

Retinal scans 105 alone may sometimes present ambiguous features, the information provided by the snapshot data 110 serves to clarify such uncertainties. For instance, changes in retinal scans 105 such as minor degradation in vasculature may occur as a result of aging rather than being indicative of a disease or a disease risk. Therefore, contemplation of retinal scans 105 in conjunction with snapshot data 110 over a series of time points may be crucial to differentiate between age-related changes and potential health risks, which may ensure more accurate risk assessment and detection of cardiovascular conditions.

CVD incidents 215 or other health events (e.g., heart attacks, strokes, or even diabetes complications) may occur at any point between the time points and may then become part of the exemplary timeline 200 and the multimodal data. Such health incidents may provide context for interpreting the multimodal data at the surrounding time points. For example, if a CVD incident 215 occurs between T2 and T3, the snapshot data 110 and the retinal scans 105 collected at T3 may show significant changes in the health status of the subject, which may directly relate to the incident that occurred after T2. It will be appreciated that the collection of retinal scans 105 and/or snapshot data 110 may occur without prior knowledge of an impending CVD incident occurring between consecutive time points. In some aspects, the CVD incident 215 may be a part of the snapshot data 110 or non-retinal data.

In some aspects, the longitudinal multimodal data including the retinal scans 105 and the snapshot data 110 over multiple time points, and/or CVD incidents 215 may collectively form a longitudinal dataset. The longitudinal dataset, which may include results from previous time points, may be combined with the recent retinal scan 205n and snapshot data 210n at Tn as part of the multimodal data for predictive analysis. Alternatively, in another aspect, multimodal data across the exemplary timeline 200 may be processed simultaneously to provide a comprehensive, up-to-date prediction for the current health status of the subject.

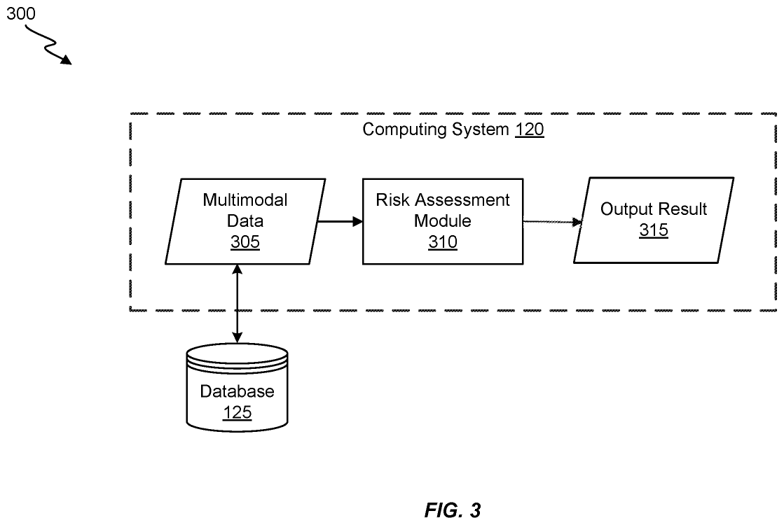

FIG. 3 illustrates an example overview of a computing system of FIG. 1 to predict the one or more diseases based on the longitudinal multimodal data of the subject in accordance with some aspects of the present disclosure. The exemplary overview 300 of the computing system 120 comprises of a risk assessment module 310 that predicts an output result 315 based on the multimodal data 305. The risk assessment module 310 may efficiently process and analyze multimodal data 305, which may also include the longitudinal multimodal data that the computing system 120 accesses from the database 125.

The risk assessment module 310 may include advanced pre-processing modules that standardize and prepare the multimodal data 305 (e.g., retinal images and snapshot data across multiple time points, and incident reports), ensuring that the multimodal data 305 is properly formatted for subsequent analysis. Following the pre-processing, the risk assessment module 310 may utilize one or more ML models to extract features from the multimodal data 305, aggregate the extracted features and make a prediction based on the aggregated feature vector. These ML models may work in tandem to integrate relevant information from different modalities, ultimately generating a comprehensive prediction of the cardiovascular health of the subject.

The risk assessment module 310 may generate one or more output result 315 based on the multimodal data 305 that may be provided to the clinician, the medical provider, or other relevant parties. The output result 315 may include predicted probability of the subject developing CVD, a predicted change, an extent of disease progression, or a predicted extent to which a given treatment would effectively treat, prevent, or manage CVD for the subject. The outlined prediction process specifically focuses on predicting CVD, but may also extend to predicting one or more other diseases such as diabetes, stroke, hypertension, glaucoma, Alzheimer's disease, neurodegenerative conditions or the like.

A profile system, integrated within the computing system 120, may govern both the multimodal data 305 and the output result 315. The profile system may manage the multimodal data 305 coming from sources such as the medical imaging systems, laboratory test results, electronic health records and/or the input from the subject, thereby forming a detailed and dynamic health profile for the subject. Authorized users including clinicians, healthcare providers, or the subject themselves may access the profile of the subject via a graphical user interface (GUI). The GUI within the computing system 120 may be used to view relevant images or health data, and potentially adjust treatment plans based on the predicted disease risks. Such flexibility may allow the users to view, update, and manage the data remotely, ensuring that critical health information is always available, whether at a clinic, at home, or on the go, thereby supporting seamless and continuous care.

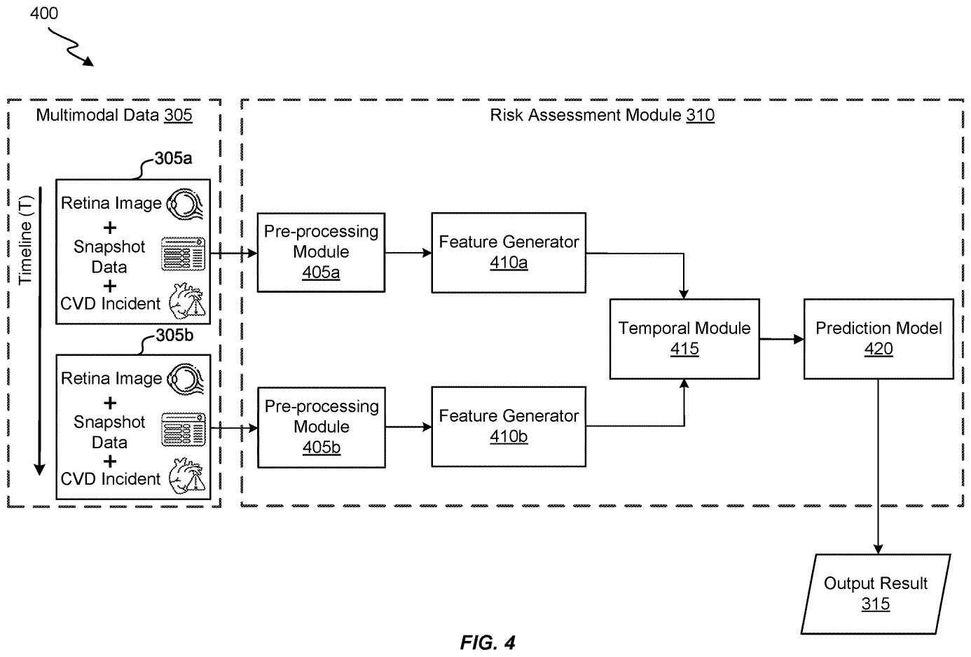

FIG. 4 illustrates a detailed block diagram 400 from the FIG. 3, predicting the one or more diseases based on the longitudinal multimodal data in accordance with some aspects of the present disclosure. Longitudinal multimodal data, where multimodal data 305a corresponding to a time point T1 and the multimodal data 305b corresponding to a later time point T2, may be used as input to the risk assessment module 310 to predict the progression of a disease risk over time. Each multimodal data 305 comprises of retinal scans 105, snapshot data 110 or CVD incident 215 over a specific time point, although it may not be necessary for a CVD incident 215 to occur at every time point of the set of time points.

The risk assessment module 310 may comprise of one or more pre-processing module 405a, 405b (which may be collectively referred to as pre-processing modules 405 and may be individually referred to as pre-processing module 405), one or more feature generator 410a, 410b (which may be collectively referred to as feature generators 410 and may be individually referred to as feature generator 410), a temporal module 415 and/or a prediction model 420.

The pre-processing module 405 may standardize the longitudinal multimodal data prior to an analysis by downstream ML models over a set of time points. Pre-processing module 405 may ensure uniformity across the retinal scans 105 of the longitudinal multimodal data by applying techniques such as normalization to adjust pixel intensities, resizing or scaling to ensure uniform dimensions, and cropping to isolate the region of interest (ROI) such as retinal blood vessels or the optic disc. Additionally, the pre-processing module 405 may enhance image quality through contrast adjustment and noise reduction filters, improving the clarity of key retinal features.

For the snapshot data 110, the pre-processing module 405 may normalize and align data across different time points, filtering out inconsistencies while preserving key temporal patterns. To improve robustness and generalization of the detailed block diagram 400, the pre-processing module 405 may perform data augmentation techniques such as rotation, flipping, translation, masking, color jitter, grayscale conversion, zooming, and adding synthetic noise. Such augmentations may ensure that the system is invariant to variations in orientation, lighting, or noise, ensuring that the pre-processing module 405 may effectively handle real-world data. Furthermore, the pre-processing module 405 may ensure that no individual data type (e.g., certain image features or clinical values) exerts an undue influence on the performance of subsequent ML models.

The detailed block diagram 400 may be configured with a single pre-processing module to handle the longitudinal multimodal data (e.g., multimodal data 305a and multimodal data 305b at different time points). Alternatively, the detailed block diagram 400 may use distinct pre-processing modules with the pre-processing module 405a dedicated to processing the multimodal data 305a and the pre-processing module 405b for the multimodal data 305b. However, the number of pre-processing modules 405 employed is not intended to limit the scope of the disclosure.

After pre-processing, the refined and pre-processed multimodal data may then be passed through subsequent one or more ML models, also termed as feature generator 410. The feature generator 410 includes extracting and quantifying key features from the pre-processed multimodal data to uncover patterns that are significant for predicting various health outcomes, including CVD risks or other age-related conditions.

From retinal modalities (e.g., retinal scans 105), extracted features may include vessel diameter, tortuosity, density, dilation, and retinal arteriovenous nicking (AV nicking), which may aid in assessing the condition of the retinal vasculature and provide insights into cardiovascular health. For non-retinal modalities (e.g., snapshot data 110 and CVD incident 215), features may be extracted from various sources such as blood pressure (systolic and diastolic), glucose levels (e.g., fasting blood glucose or HbA1c), cholesterol levels (total cholesterol, LDL, HDL, triglycerides), body mass index (BMI), medical imaging data (e.g., X-rays, CT scans, MRIs of any body part apart from retina) and lifestyle factors like age, gender, smoking status, physical activity, dietary habits, alcohol consumption, family history and/or input from the subject. Additionally, the presence of comorbidities like hypertension, diabetes, or metabolic syndrome further enriches the feature set, providing a comprehensive view of the health status of the subject and enabling accurate risk predictions.

The feature generator 410 may work on the pre-processed multimodal data jointly or separately. For separate processing, convolutional neural networks (CNNs) or transformer-based networks such as vision transformers (ViT) may be employed to extract visual features from the retinal scans 105. Such models may capture spatial relationships and patterns critical for predicting retinal degradation. For non-retinal modalities, which include clinical, demographic, behavioral, and medical imaging data, various models may be used for feature extraction. For textual data, models like BERT or ROBERTa may be employed to capture the subtleties of the health status of the subject. For imaging data, convolutional neural networks (CNNs) or other specialized models are used to extract relevant features from the scans (e.g., MRIs, CT scans etc.). Other models like decision trees, support vector machines (SVM), or gradient boosting may be employed to learn patterns related to risk factors such as age, smoking, or blood pressure.

Alternatively, for joint processing, the feature generator 410 may incorporate multimodal models such as UNITER or ViLT to analyze retinal scans 105, snapshot data 110 and/or CVD incident (if any) in tandem. Such models may process visual and textual information within a shared space, generating a feature vector that may also capture the relationship between different modalities.

The temporal module 415 aggregates a set of feature vectors by merging the feature vectors derived from the feature generators 410a and 410b, where each feature vector of the set of feature vector may correspond to a different time point. Aggregation process may ensure the preservation of the temporal relationships between the multimodal data at each time point. To capture global trends and local changes, the temporal module 415 may normalize the set of feature vectors and then perform sum and difference operations on such normalized set of feature vectors. By performing normalization, the temporal module 415 may ensure that each feature vector of the set of feature vectors may be treated consistently, irrespective of scale differences. Further, the sum operation may combine information from all previous time points to reflect long-term patterns, while the difference operation may highlight short-term fluctuations between consecutive time points resulting in a cumulative vector and a differential vector. Such cumulative and differential vectors may then be concatenated together to form a unified representation (herein, referred to as the temporal feature vector) of both the cumulative (global) and differential (local) features. The temporal module 415 may process the temporal feature vector using advanced models like recurrent neural networks (RNNs), long short-term memory (LSTM) networks, or temporal convolutional networks (TCNs).

The temporal feature vector from the temporal module 415 may be forwarded to the prediction model 420. The prediction model 420 may leverage the temporal feature vector (e.g., the aggregated vector) to make predictions about the health status of the subject including risk scores for one or more diseases particularly CVD risk scores, potential disease progression or the effects of potential interventions. The training set T comprises of n training samples represented as,

$$T = {\{ {x}_{i} , {y}_{i}^{rgr} \}}_{i = 1}^{n} ,$$

where xᵢ denotes the temporal future vector and

$${y}_{i}^{rgr}$$

denotes the continuous regression output (e.g., risk score, predicted trend or the like). The prediction model 420 may be defined as the function of f_{θ}: x→y_{rgr}, where x is the input temporal feature vector and y^{rgr} is the corresponding prediction output.

The prediction model 420 may be a single model that may be based on algorithms such as deep learning models (DNNs), neural networks, decision trees (e.g., random forests), gradient boosting machines (XGBoost, LightGBM), support vector regression (SVR), k-Nearest Neighbors (k-NN), multilayer perceptron (MLP), Bayesian networks, or Neural ODEs. Such algorithms may capture complex, non-linear relationships in the longitudinal multimodal data. Alternatively, the prediction model 420 may be a set of sub-models, each focusing on specific aspects of the longitudinal multimodal data. The set of sub-models may include but are not limited to Cox-based risk model for survival analysis, incident-based risk model (e.g., predicting the likelihood of disease onset), aging model, patient information model or the like, to track the health progression over time.

After processing the temporal feature vector, the prediction model 420 may generate a disease-prediction metric associated with the output result 315 that predicts the subject's likelihood of having or developing CVD. The disease-prediction metric may include a binary prediction (e.g., a high risk or a low risk), a class (e.g., representing a predicted likelihood of a subject having or developing CVD), a numeric prediction (e.g., a probability score representing a predicted probability of a subject currently having CVD, a predicted probability of a subject developing CVD within a predefined time period, a predicted degree of change of a subject's CVD within a predefined time period, etc.). The output result 315 may then be presented on the GUI, where the clinician, the healthcare provider or the subject may review and interpret the CVD risk score. Based on the CVD risk score, informed decisions about the treatment plan or management strategies associated with the subject may be made.

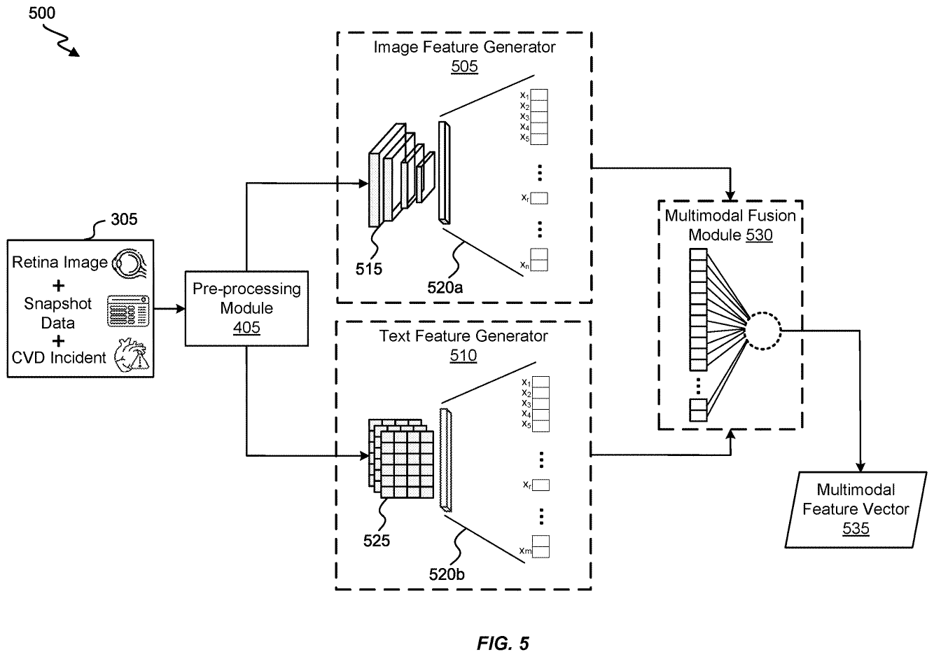

FIG. 5 illustrates a block diagram 500 depicting a process of aggregating features from the longitudinal multimodal data into a multimodal feature vector in accordance with some aspects of the present disclosure. Block diagram 500 begins with the multimodal data 305 that may be from any time point of the set of time points (e.g., T1, T2, . . . or Tn), which may be pre-processed by the pre-processing module 405. The pre-processing module 405, in addition to standardizing the data, may also separate the multimodal data 305 into different modalities. In some aspects, the modalities may include image-based modalities (e.g., retinal scans 105, and medical imaging data including MRIs, CT scans of any other body part apart from retina and/or CVD reports from the non-retinal data) and text-based modalities (e.g., blood tests, blood glucose levels or the like from the non-retinal data). The pre-processed data may then be sent to two separate feature generation pathways via the one or more ML models including a first ML model (also termed, hereafter, as an image feature generator 505) and a second ML model (also termed, hereafter, as a text feature generator 510).

The image feature generator 505 corresponds to the image-based modalities that may extract features via an image feature extractor 515, where the image feature extractor 515 may include convolutional neural networks (CNNs) or pre-trained CNN models such as AlexNet, VGGNet, ResNet, GoogLeNet, or DenseNet. The image feature extractor 515 may be adapted by modifying the final layer to generate the appropriate feature vectors for the specific task. The CNNs may be fine-tuned using a dataset of target retinal scan to enhance their effectiveness in capturing relevant features from the image-based modalities.

In addition to the CNN-based architecture, more specialized models may be employed for imaging tasks. For instance, Attention U-Net may be integrated to focus on relevant regions within the images or scans by incorporating attention mechanisms. DeepLab may also be used to leverage atrous convolutions, facilitating dense feature extraction and improved segmentation across the images. Furthermore, advanced techniques such as Vision Transformers (ViT) may be employed as an alternative to CNNs. ViT models generate patch embeddings from non-overlapping image patches and utilize multi-head, self-attention mechanisms to capture long-range dependencies and relationships between different image regions. The resulting transformer layers may then be used to generate a first embedding vector, also termed as image embedding vector 520a.

The text feature generator 510 corresponds to text-based modalities, where the input may be in the form of a matrix 525. Rows of the matrix 525 may represent different time points or clinical instances and columns may represent various clinical features (e.g., lab results, vital signs, etc.). The matrix 525 may represent a common format for organizing text-based modalities. Other possible formats may include tabular format, sequential format, which emphasize the temporal trends. Sparse matrices may also be used to represent incomplete clinical data, where missing values may be handled appropriately.

The matrix 525 may be converted into a second embedding vector, also termed as a text embedding vector 520b using several techniques such as CNNs, where convolutional filters move across the matrix to capture local patterns and relationships between clinical features over time. Pooling operations may then be applied to reduce dimensionality and the resulting matrix may flatten into a one-dimensional vector. The flattened vector represents the final form of the text embedding vector 520b. Alternatively, Recurrent Neural Networks (RNNs) or Long Short-Term Memory (LSTM) networks may process the matrix 525 as sequential data to capture temporal dependencies between the set of time points and generating a final hidden state as the text embedding vector 520b. Autoencoders may also be used to compress the matrix 525 into a lower-dimensional representation that may serve as the text embedding vector 520b.

The block diagram 500 is designed to maintain separate pathways for image and text feature extraction, allowing for more precise and specialized feature generation from each modality. The image embedding vector 520a may have a similar or different length compared to the text embedding vector 520b. The image embedding vector 520a may span from x₁ to xₙ, where xᵣ represents any intermediate vector point. Similarly, the text embedding vector 520b may span from x₁ to xₘ, with x, being any middle term. The difference in lengths of both the embedding vectors may allow for flexibility in the representation of features extracted from both image-based modalities and text-based modalities,

The image embedding vector 520a and text embedding vector 520b may then be passed to a third ML model of the one or more ML models (also termed, hereafter, as a multimodal fusion module 530). The multimodal fusion module 530 may aggregate or fuse the two embedding vectors to combine the complementary information from the image and text modalities. The output of the multimodal fusion module 530 may be a feature vector 535, which may vary in length depending on the dimensions of the input embedding vectors. The length of the feature vector 535 may be the sum of the lengths of the image embedding vector 520a and the text embedding vector 520b, or it may be a fixed length that may be determined by the fusion process to accommodate the combined information. The feature vector 535 may further be utilized for downstream tasks such as prediction or risk assessment.

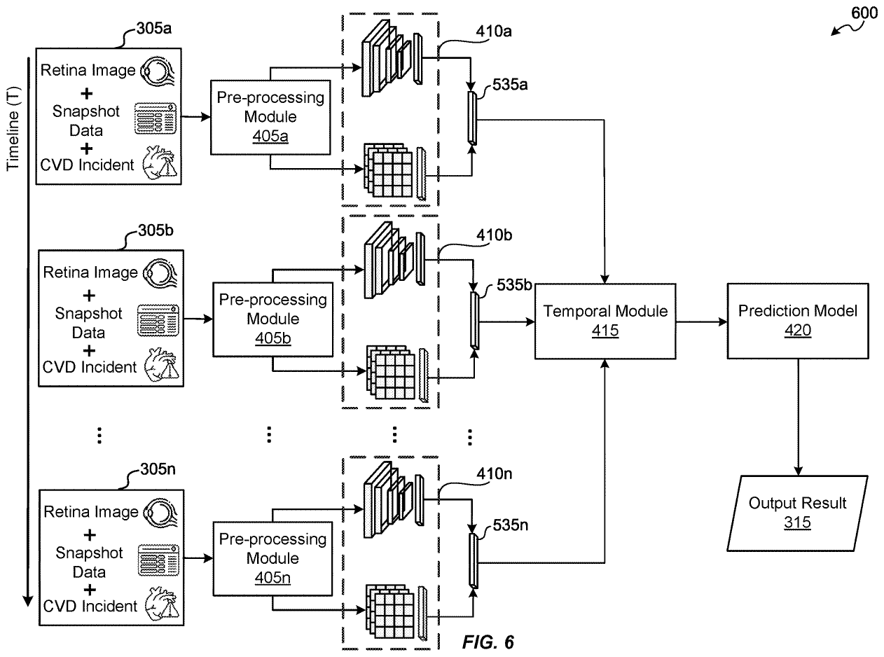

FIG. 6 illustrates an example block diagram 600 depicting a process of aggregating a set of feature vectors into a temporal feature vector to make predictions in accordance with some aspects of the present disclosure. Exemplary block diagram 600 may represent the multimodal data 305 from the set of time points such as from T1 to Tn. The multimodal data 305a, 305b, . . . , 305n may be pre-processed by the pre-processing modules 405a, 405b, . . . , 405n.

The pre-processing module 405 may send the pre-processed multimodal data to the feature generator 410a, 410b, . . . , 410n. In some aspects, each of the feature generator 410 may comprise of the image feature generator 505 and the text feature generator 510, where the data coming from the pre-processing modules 405 may be handled separately. One such example is illustrated in FIG. 4, where the image feature generator 505 extract features from the image-based modalities and the text feature generator 510 extracts features from the text-based modalities.

In some aspects, the feature generator 410 may be the multimodal model that may extract features from both modalities including the image-based modalities and the text-based modalities. Such models may leverage techniques like attention mechanisms to align and merge image and text features, learning the relationships between the two and ensuring that complementary information from both types may be captured. The feature vectors 535a, 535b, . . . , 535n, which may be collectively referred to as a set of feature vectors 535 and may individually be referred as feature vector 535, may then be created by combining the extracted image and text features.

The set of feature vectors 535 associated with the set of time points (e.g., T1, T2, . . . , Tn) may be aggregated to generate a temporal feature vector using the temporal module 415. The fusion process may allow the system to capture dynamic changes and also the temporal relationships across different modalities over time. In some aspects, the fusion process may be a late fusion as disclosed herein, where the temporal module 415 may apply operations such as sum and difference to capture global trends and local changes across the multiple time points. Before any operation may be applied, the set of feature vectors are normalized that may ensure consistency across the multiple time points. The normalization may ensure that the changes in the set of feature vectors 535 across multiple time points are comparable, which may prevent any time point to dominate due to a scale difference. Regarding the global trends, the sum operation can be performed to compute the cumulative vector V_{Tn} represented as: V_{Tn}=V_{T(n-1)}+V_{Tn}, where V_{T(n-1)}=V_{T1}+V_{T2}+ . . . +V_{T(n-1)} represents the cumulative vector from the previous time points. Here, V_{T1}, V_{T2}, . . . , V_{Tn} corresponds to the set of feature vectors 535a, 535b, . . . , 535n that are normalized at time points T1, T2, . . . , Tn. Similarly, for the local changes, the differential vector between the consecutive time points can be computed as:

$${V}_{Tn}^{′} = {V}_{Tn} - {V}_{T ( n - 1 )} .$$

The cumulative vector V_{Tn} and differential vector

$${V}_{Tn}^{′}$$

can track long-term global trends and short-term fluctuations between the adjacent time points. Both trends may show accurate prediction, as the global trends provide a comprehensive understanding of overall changes across the multiple time points, while the local changes may highlight more immediate variations or anomalies that may be decisive for the system's decision-making process.

The temporal module 415, which may utilize techniques such as concatenation, weighted averaging, or advanced models like RNNs, LSTMs, or temporal convolutional networks (TCNs), may concatenate the cumulative vector V_{Tn} and differential vectors

$${V}_{Tn}^{′}$$

to generate the temporal feature vector. The RNN or LSTM can sequentially update such vectors by remembering previous time points and adjusting the temporal feature vector based on incoming multimodal data. The temporal feature vector is then fed into the prediction model 420 to generate the prediction (e.g., output result 315). Alternatively, the fusion process may be early fusion, where the multimodal data 305 from different time points may be combined before feature extraction. Early fusion may allow the temporal module 415 to integrate different modalities (such as images and text) at the input level and may enable the system to learn complex relationships between them from the outset.

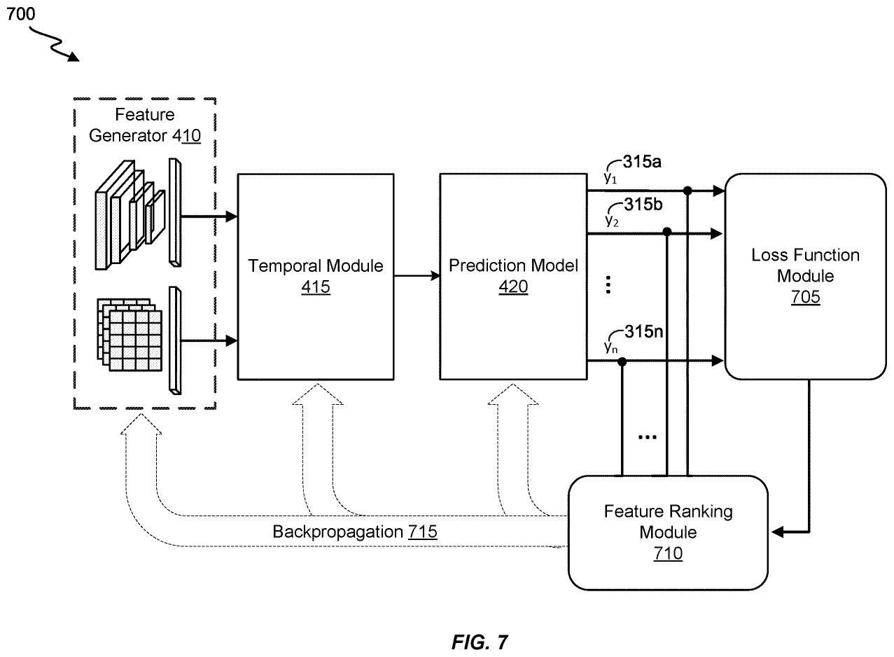

FIG. 7 illustrates an exemplary training of one or more machine-learning (ML) models, utilizing a loss function in accordance with some aspects of the present disclosure. The loss function may be implemented through a loss function module 705, which may train one or more ML models including the feature generator 410, the temporal module 415 and the prediction model may collectively serve as a single model and may be trained together in an end-to-end manner. The output results 315a, 315b, . . . , 315n from the prediction model 420 may be fed into the loss function module 705 and a feature ranking module 710 to allow the adjustment in the associated ML models through backpropagation 715.

The loss function is a mathematical method that may evaluate the accuracy of predictions by quantifying the difference between the predicted outputs and the actual outcomes. The primary goal of the loss function is to upgrade the models by providing a scalar value that may indicate how well each model's predictions match the true values. In some aspects, the loss function module 705 may calculate discrepancies between the output results 315 and true outcomes, where the output results 315 may involve predicting risk scores for CVD, the likelihood of disease progression, or other clinical outcomes. After computing the loss, the predicted output results from the prediction model 420 may be sent to a feature ranking module 710.

A feature ranking module 710 may then use the loss information from the loss function module 705 to rank the features according to their impact on the predictions. The feature ranking module 710 may assign weights wᵢ to each output result (yᵢ→L) based on the loss values, where i=1, 2, . . . , n. Features that significantly impact accuracy of the one or more ML models may receive higher weights such as those related to the presence or severity of CVD or contributors that may promote CVD risk including hypertension, elevated cholesterol, or smoking. The assigned weights may then be applied during training and/or when processing the multimodal data 305 in a future time point.

Techniques such as attention mechanisms or permutation importance may further refine the ranking process by dynamically adjusting feature importance during training. The attention mechanism may assign an attention score at to each feature that reflects its relevance during a given prediction. The weight update for each feature may then be represented as:

$${ω}_{i}^{new} = {ω}_{i}^{old} + {α}_{i} \cdot {Δω}_{i} , where {ω}_{i}^{new}$$

is the updated weight,

$${ω}_{i}^{old}$$

is the current weight, aᵢ is the attention score for feature i and Δωᵢ is the gradient of the loss function with respect to feature i. Similarly, permutation importance may identify the key features (or output results) by evaluating the drop in model performance when the feature is randomly permuted. If permuting a feature causes a significant decrease in model performance, that feature is deemed more important. The importance of each feature may be represented as:

$${I}_{perm} ( {x}_{i} ) = \frac{1}{N} {\sum }_{j = 1}^{N} ( L ( {f}_{xi} ) - L ( {f}_{xi}^{perm} ) ) ,$$

where Iₚₑᵣₘ(xᵢ) is the permutation importance for feature xᵢ, L(fₓᵢ) is the loss of the model using the original feature, and

$$L ( {f}_{xi}^{perm} )$$

is the loss atter permuting feature xᵢ. The higher the drop in loss, the more important the feature is. Therefore, the feature ranking module 710 may leverage permutation importance along with the loss information from the loss function module 705 to rank the notable features.

During the backpropagation 715, the gradients of the loss with respect to each model's parameters (including weights and/or biases of the feature generator 410, the temporal module 415 and the prediction model 420) may be computed and updated to reduce the overall loss. Therefore, the backpropagation 715 may allow the models to progressively adjust and improve the ability to distinguish between different sources of retinal degradation, improving the ability of the models to make accurate predictions and risk assessments related to disease and aging.

Moreover, the loss function module 705 may also be used to distinguish between retinal degradation caused by aging and by disease risk. By leveraging temporal aspect of the longitudinal multimodal data to track natural aging progression and to focus on disease-specific retinal features, the loss function module 705 may ensure that the models may learn to differentiate between the two types of retinal degradation. Such differentiation may improve the accuracy of disease risk predictions by curtailing confusion between normal aging and pathological changes in the retina that may be indicative of a disease risk.

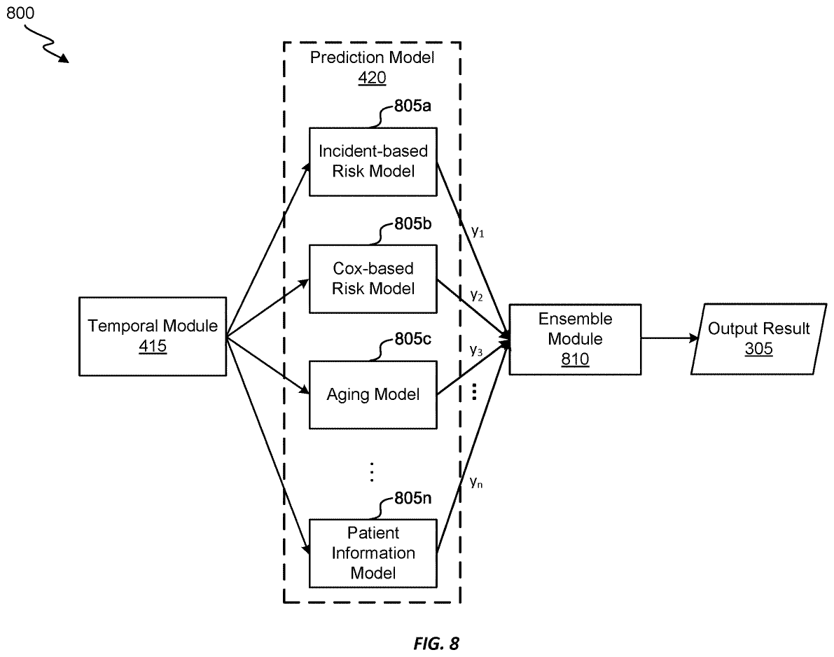

FIG. 8 illustrates prediction of the one or more diseases using a set of sub-models to separately process one or more factors associated with the temporal feature vector in accordance with some aspects of the present disclosure. The longitudinal multimodal data including the retinal scans 205, snapshot data 210 or any major health incident (e.g., CVD incident, diabetic complications or the like) may all be processed and integrated over time by the temporal module 415 to generate the temporal feature vector. The temporal module 415 passes on the temporal feature vector to be fed into the prediction model 420. In some aspects, the prediction model 420 may be a set of sub-models that may include but are not limited to an incident-based risk model 805a, a Cox-based risk model 805b, an aging model 805c, . . . , or a patient information model 805n, which hereafter may be collectively referred to as set of sub-models 805.

Each sub-model within the set of sub-models 805 may analyze distinct factors such as disease history, clinical data, aging progression, and patient-specific characteristics, allowing for a more holistic and accurate assessment of disease risk particularly a CVD risk. For instance, the incident-based risk model 805a may analyze the history of disease incidents such as previous CVD or diabetic episodes within the context of the longitudinal multimodal data. The incident-based risk model 805a may focus on how past incidents may influence the likelihood of future events, thereby integrating both the sequence of health events and their impact on disease progression.

The Cox-based risk model 805b may evaluate the likelihood of future health outcomes over time based on clinical and demographic factors such as age, gender, body mass index, cholesterol levels, and other biomarkers. By incorporating the temporal aspects of the data, the Cox-based risk model 805b may account for changes in risk factors over time. The aging model 805c may analyze the natural aging progression of the subject by focusing on the physiological changes over time (e.g., increased blood pressure or kidney function decline) that may affect health outcomes. Further, the patient information model 805n may integrate patient-specific data such as medical history, lifestyle factors (e.g., smoking, diet, exercise) and genetic information, all within the longitudinal multimodal data context. By evaluating how such factors evolve over time, the patient information model 805n may predict how changing circumstances of a subject influence their disease risk.

In addition to the incident-based risk model 805a, Cox-based risk model 805b, aging model 805c, . . . , and patient information model 805n, many other sub-models may be present in between. For instance, a genetic risk model that may evaluate genetic markers contributing to a predisposition to diseases such as CVD, diabetes, hypertension, neurodegenerative diseases or the like. Additionally, a psychosocial risk model may factor in mental health and social determinants, which may have a profound impact on overall well-being of the subject. Further, a medication risk model may analyze the effects of current medications, which may help predict how the medications may influence disease outcomes especially when interacting with other health factors. It will be appreciated that the set of sub-models 805 may include any suitable model or combination of models. The type of sub-models and the number of sub-models within the set of sub-models 805 may not limit the invention.

Predictions (for example) yᵢ from the set of sub-models 805 may then be fed into an ensemble module 810, which comprises of the ensemble model. The ensemble model may integrate the predictions by applying weighted factors to generate a final, weighted output result that reflects an accurate disease risk assessment. Predictions yᵢ may then be assigned a weight wᵢ based on its relevance to the temporal feature vector of the subject. The final prediction is calculated as a weighted sum of each sub-model outputs:

$${y}_{i} = {\sum }_{i = 1}^{n} {ω}_{i} \cdot {y}_{i} .$$

For example, if the aging model 805c detects significant retinal degradation due to natural aging rather than CVD, the weight ω_{aging} for the aging model prediction will be higher than other sub-models. Therefore, by effectively combining the outputs from the set of sub-models 805, the ensemble module 810 ensures that pertinent information may be prioritized. The weights assigned to each sub-model of the set of sub-models 805 may be dynamically adjusted during the training process using the loss function (e.g., mean squared error or cross-entropy loss) that may evaluate the accuracy of the combined prediction. The weights are updated through backpropagation to reduce the error as also illustrated in FIG. 7.

The ensemble module 810 may provide a more comprehensive, multi-faceted prediction of the overall CVD risk of the subject, ensuring that the final risk score accounts for all available information and reflects the complexity of the health status of the subject. Therefore, the output result 315 may combine insights from various aspects of the longitudinal multimodal data, such as past incidents, clinical factors, aging progression, and patient-specific information, providing a more holistic and accurate disease risk assessment.

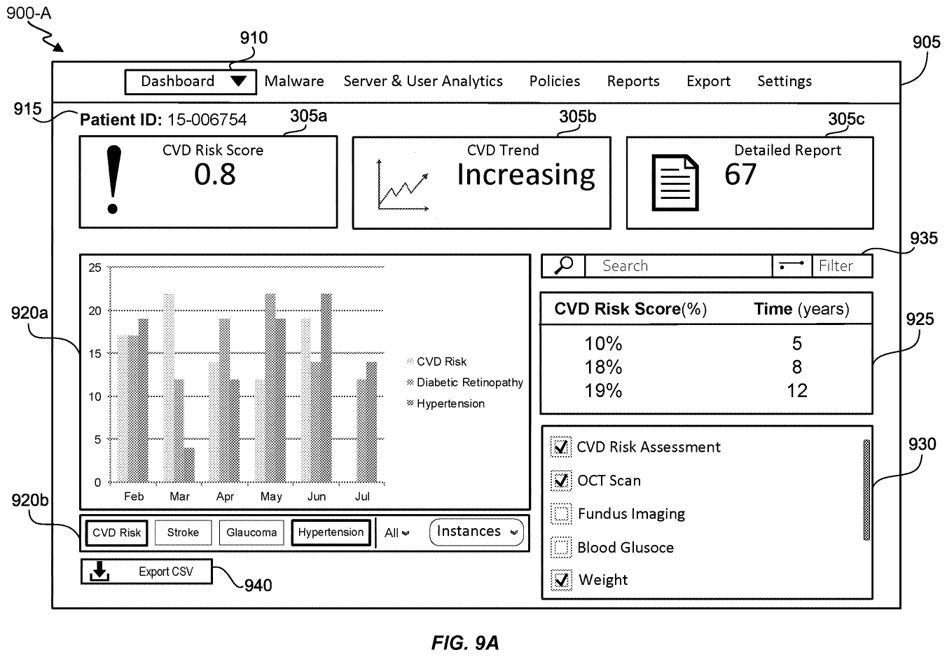

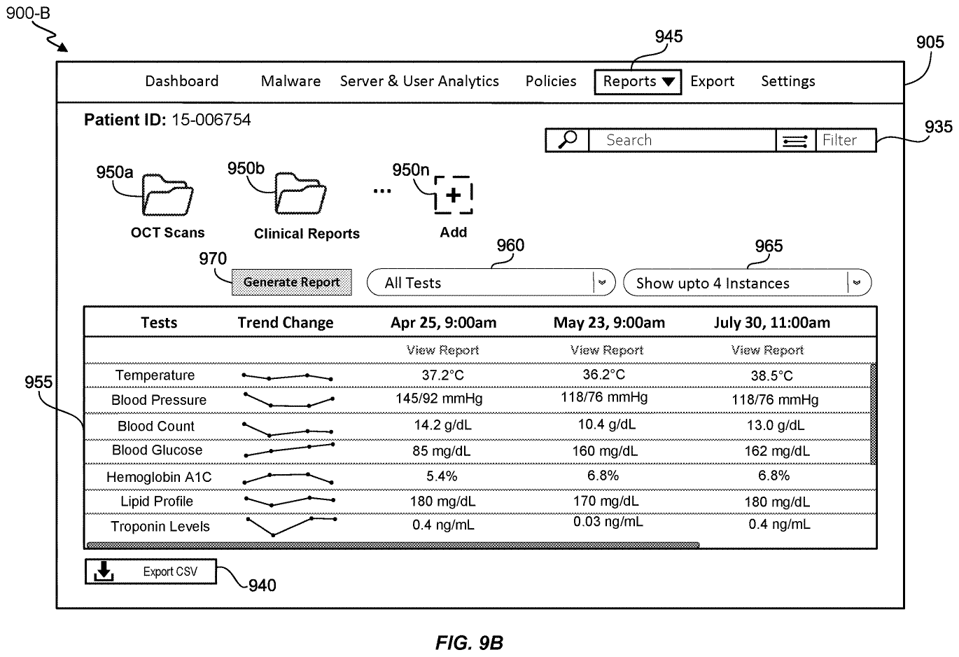

FIG. 9A shows an illustrative example of a first graphical user interface (GUI) 900-A representing a dashboard for tracking and accessing the one or more diseases in accordance with some aspects of the present disclosure. The first GUI 900-A illustrates the profile of the subject under profile ID 915. The profile ID 915 may represent a unique ID of the subject that may serve as a key to access the profile of the subject. The profile of the subject may allow continuous monitoring and updating of the data associated with the subject.

The top section features a toolbar 905 that may allow access to various sections related to the health profile of the subject including options for malware analysis, server/user analytics, policies, reports, exports, and settings. Currently, the user is on the dashboard 910, where a risk report is displayed. Below the toolbar 905, the output results 315a, 315b, 315c of the subject may be displayed. The output results 315 may provide insight into the health status of the subject and the prediction of the one or more disease risks particularly CVD risks. The output result 315a, 315b and 315c illustrates a CVD risk score, a trend of CVD risk overtime and a detailed report respectively. The detailed report may include test results, relevance to the subject's disease risk and associated recommendations.

The first GUI may also illustrate a risk prediction graph 920a that maps one or more disease risks associated with the subject. The risk prediction graph may show fluctuations of CVD, diabetic retinopathy, hypertension risks and the like over the set of time points. Associated with the risk prediction graph 920a may be a risk bar 920b that may allow the user to select which disease risks to display, providing an option to track the risk of multiple diseases across different instances. The selection in the risk bar 920b may influence the risk prediction graph 920a by mapping the risks of the selected diseases over multiple instances (e.g., 2 instances, 4 instances, 6 instances etc.).

The first GUI 900-A may also display a risk prediction table 925 along the risk prediction graph 920a. The risk prediction table 925 may be a tabular form of the risk prediction graph 920a, but over larger instances of time. In some aspects, the risk prediction table may display the predicted risk score for CVD over several years, providing a clear snapshot of how the risk profile of the subject may evolve over time based on the longitudinal multimodal data available.

The dashboard of the first GUI may also comprise of a test tracker 930, which may allow the user to view and keep track of the test that has already been conducted such as OCT scans, fundus imaging, blood glucose tests, and weight measurements. The test tracker 930 may be designed to display only the most recent tests disregarding any previous ones. The test tracker 930 may also inform the users whether all obligatory tests have been performed or if any are pending. In another aspect, the test tracker 930 may enable users to selectively designate which tests may be used for generating disease risk predictions, offering a more tailored and precise analysis.

Additionally, users may search for specific queries related to the subject using a search filter 935. The search filter 935 may enable quick and easy access to particular information or sections within the profile of the subject. Export CSV 940 may provide an option to export the report in CSV format. The report may also be exported in any other format such as PDF, Excel, JSON, and XML. Such multiple export options may enable the user to save or share the health status of the subject for further review or analysis.

FIG. 9B shows an illustrative example of a second GUI 900-B representing a report section based on the longitudinal multimodal data in accordance with some aspects of the present disclosure. Report section 945 of the toolbar 905 shows the clinical data longitudinally as to track the health of the subject over time.

Top section of the second GUI 900-B comprises records of OCT scans 950a, clinical reports 950b that may be stored across multiple time points. Such records may comprise of reports of various laboratory or clinical tests. In addition to OCT scans 950a and clinical reports 950b, users may add other medical records through an add icon 950n such as MRI or CT scan reports for detailed organ imaging, ECG records for monitoring heart rhythms or X-ray and ultrasound reports for diagnosing bone fractures, lung conditions, or assessing organ health. Such records may allow for a more comprehensive collection of the subject's medical history and test results.

The second GUI 900-B may also illustrate a longitudinal data table 955 that may present a tabular view of the clinical or laboratory test. Each column represents a snapshot of results at a particular instance (e.g., Apr 25, at 9:00 AM) with corresponding values like temperature, blood pressure, blood count, glucose levels and other critical biomarkers. The trend change for each parameter may also be displayed that allow clinicians to quickly assess whether the condition of the subject is improving or deteriorating over time. Users may also view detailed reports for each test result by selecting a View Report link under any time point.

Above the longitudinal data table 955, a test filter 960, an instance filter 965 and a generate report 970 button may be present. The test filter 960 dropdown may allow the users to select which specific tests to display in the longitudinal data table 955. For instance, users may filter to show only tests related to blood glucose and blood pressure, apart from other irrelevant tests. The instance filter 965 may allow the user to select how many time instances should be shown in the longitudinal data table 955. The second GUI 900-B may display data from just the last two clinical visits or up to any number of instances. The generate report 970 button may generate a comprehensive report based on the selected tests and time instances. The generated report may appear in a format similar to the first GUI 900-A or may follow an alternative format depending on the preference or need of the user.

The search filter 935 and export CSV 940 functionalities may allow for quick query lookups and easy export of the data in formats such as CSV, PDF, Excel, JSON, and XML for further processing, sharing, or archiving. The first GUI 900-A and the second GUI 900-B may enable the users to track health progress, analyze trends across different health parameters, and generate detailed, customizable reports to enhance both diagnosis and monitoring over multiple time points.

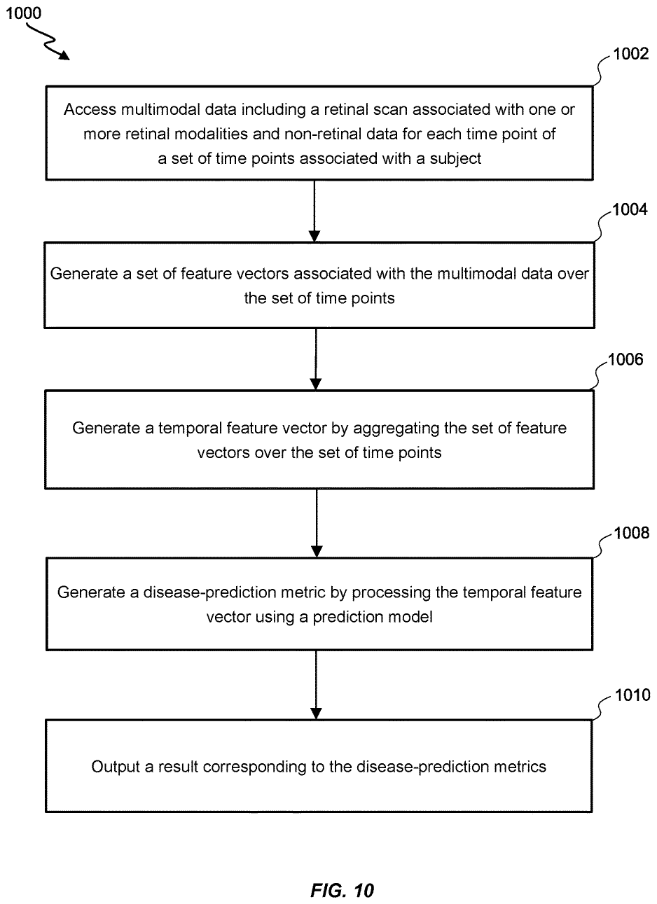

FIG. 10 illustrates an exemplary workflow 1000 to predict the one or more diseases using the longitudinal multimodal data in accordance with some aspects of the present disclosure. The blocks in the exemplary workflow 1000 are illustrated in a specific order, while the order may be modified, for example, some blocks may be performed before others, and some blocks may be performed simultaneously. The block may be performed by hardware, software, or a combination thereof.

At block 1002, the risk assessment module 310 within the computing system 120 may access the multimodal data 305 of the subject. The multimodal data 305 may include a retinal scan 105 and snapshot data 110 for each time point of the set of time points. The retinal scan 105 may be derived from one or more retinal modalities and the snapshot data 110 may correspond to non-retinal data. The multimodal data 305 may be collected all the time points of the set of time points (e.g., from T1 to Tn).

At block 1004, one or more feature generators 410 may generate a set of feature vectors associated with the multimodal data 305 of the set of time points. In some aspects, each feature vector of the set of feature vectors may be generated using the image feature generator 505 (e.g., the first ML model) and the text feature generator 510 (e.g., the second ML model). The image feature generator 505 may extract features from the image-based modalities to generate the first embedding vector or image embedding vector 520a and the text feature generator 510 may extract features from the text-based modalities to generate the second embedding vector or text embedding vector 520b. The first and the second embedding vector may be aggregated to form feature vector of the set of feature vectors 535.

At block 1006, the temporal module 415 may generate the temporal feature vector by aggregating the set of feature vectors 535 associated with the set of time points. A disease-prediction metric may then be generated based on the temporal feature vector, at block 1008. The disease-prediction metric may include but is not limited to a binary prediction (e.g., high or low risk), a class (e.g., likelihood of having or developing CVD), or a numeric prediction (e.g., probability score for current or future CVD risk, or the degree of change in CVD over time).

At block 1010, the output result 315 is generated corresponding to the disease-prediction metric that may include a risk score for the subject's likelihood of experiencing specific CVDs (e.g., heart attack, stroke) within a defined time period. The output result 315 may further include predictions of current or future CVD presence, severity, or specific types. Additionally, the output result 315 may also predict one or more other diseases such as diabetic retinopathy, age-related macular degeneration, glaucoma, stroke, Alzheimer's, Parkinson's, chronic kidney disease, and systemic inflammatory diseases like lupus and rheumatoid arthritis.

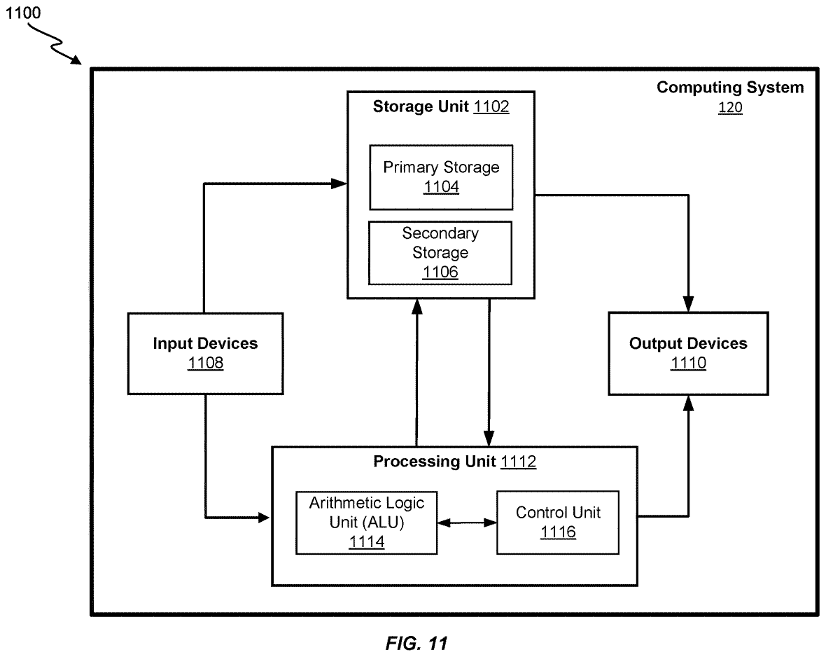

FIG. 11 illustrates an exemplary block diagram 1100 of a computing system 120 in which various aspects of the disclosed techniques may be executed. The functionality described herein may be performed, at least in part or a combination of one or more hardware or software logic components. For example, the techniques described above for predicting one or more disease risks, particularly CVD risk for a subject using temporal longitudinal multimodal data (e.g., retinal scans and snapshot data over multiple time points) by leveraging machine-learning models may be implemented in computer-executable instructions. The instructions may be executed by processing unit 1112 that may be a combination of an arithmetic logic unit 1114 that performs arithmetic and logical operations and a control unit 1116 that may help in execution of the instructions. The control unit 1116 may direct and coordinate the operation of the processor with other parts of the computer by synchronizing data flow between different components of the processing unit 1112. It may manage the flow of instructions and data between various components. The control unit 1116 may decode an operation code (opcode) and may convert them into control signals to coordinate how data moves within the processing unit 1112. The control unit 1116 may regulate execution units such as the arithmetic logic unit 1014 and the flow of data to primary storage 1104 and secondary storage 1106.

To provide additional context for various aspects thereof, FIG. 11 and the following description are intended to provide a brief, general description of the computing system 120 in which the various aspects may be implemented. While the description above is in the general context of computer-executable instructions that may run on one or more computing systems, those skilled in the art will recognize that a novel implementation also may be realized in combination with other program modules and/or as a combination of hardware and software. The computing system for implementing various aspects includes a processing unit 1112 having one or more processors (also referred to as microprocessors), a computer-readable storage medium (where the medium is any physical device or material on which data may be electronically and/or optically stored and retrieved) such as a data storage unit 1102 (computer readable storage medium/media also include magnetic disks, optical disks, solid state drives, external memory systems, and flash memory drives), and a system bus. The data storage unit 1102 may have a primary storage 1104 and a secondary storage 1106 as described here in. The primary storage 1104 and the secondary storage 1106 may differ in speed of access, connection with the computer's processor and data retrieval speeds. Primary storage 1104 may often be directly connected to the computer's processor, boasts rapid data retrieval speeds. In contrast, secondary storage 1106 may be designed for long-term storage and may have slower access times.

The computing system may include various microprocessors, such as single-processor, multi-processor, single-core, and multi-core units for processing and storage. Additionally, experts in the field recognize that the innovative system and methods may be applied to other computing configurations, including minicomputers, mainframe computers, personal computers (such as desktops, laptops, and tablet PCs), handheld computing devices, microprocessor-based consumer electronics, and similar systems. These systems may be interconnected with one or more associated devices.

In some aspects, the computing system may include one of several computers employed in a datacenter and/or computing resources (hardware and/or software) in support of cloud computing services for portable and/or mobile computing systems such as wireless communications devices, cellular telephones, and other mobile-capable devices. Cloud computing services, include, but are not limited to, infrastructure as a service (IaaS), platform as a service, software as a service (SaaS), storage as a service (StaaS), data as a service (DaaS), security as a service and APIs (application program interfaces) as a service. In some instances, data storage unit 1102 may include computer-readable storage (physical storage) medium such as a volatile memory (e.g. random-access memory (RAM) also termed as the primary storage 1104) and a non-volatile memory (e.g., ROM). A basic input/output system (BIOS) may be stored in the non-volatile memory and includes the basic routines that facilitate the communication of data and signals between components within the computing system, such as during startup. The volatile memory also includes a high-speed RAM such as static RAM for caching data.

As an illustrative example (without limiting the scope), the data storage unit 1102 may include program modules. These modules may encompass client applications, web browsers, mid-tier applications, relational database management systems (RDBMS), and more. Additionally, the data storage unit 1102 holds program data and an operating system. The operating system running may include (for example) Microsoft Windows®, Apple Macintosh®, or Linux. Furthermore, commercially available UNIX®-like operating systems (such as GNU/Linux variants and Google Chrome OS) and mobile operating systems (like iOS, Windows® Phone, Android OS, BlackBerry® OS, and Palm® OS) are part of this landscape. Notably, portions of the operating system, program modules, and program data may be cached in the storage unit 1102—both volatile memory (e.g., RAM) and non-volatile memory (e.g., ROM). This flexibility allows the disclosed architecture to be implemented using a variety of commercially available operating systems or combinations thereof (including virtual machines).

In some other examples, the computing system may have additional features or functionality. For example, the computing system may also include additional data storage devices (removable and/or non-removable) such as, for example, magnetic disks, optical disks, or tape. Computer-readable media may include, at least, two types of computer-readable media, namely computer storage media and communication media. Computer storage media may include volatile and non-volatile, removable, and non-removable media implemented in any method or technology for storage of information, such as computer readable instructions, data structures, program modules, or other data.

The storage media of the computing system may also include removable storage, and non-removable storage. EEPROM, flash memory or other memory technology, CD-ROM, digital versatile disks (DVD) or other optical storage, magnetic cassettes, magnetic tape, magnetic disk storage or other magnetic storage devices, or any other non-transmission medium that may be used to store the targeted information and which computing system may access are examples of computer storage media in addition to RAM and ROM. Additionally, the computer-readable media might have computer-executable instructions that the processing unit 1112 may use to carry out the different tasks and/or operations mentioned in this article. In contrast, communication media may embody computer readable instructions, data structures, program modules, or other data in a modulated data signal, such as a carrier wave, or other transmission mechanism.

One or more input devices, such as a keyboard, mouse, pen, voice input device, touch input device, etc., may also be included in the computing system. There might also be one or more output devices 1110, including speakers, printers, displays, and so on. These devices are not covered in detail here because they are well known in the field. To establish communication, the computing system may further have one or more network interfaces. This would enable the computing system to communicate with other systems or devices, for example, over a network. Both wired and wireless networks could be a part of these networks. Here, the computing system is one example of a suitable device or system and is not intended to suggest any limitation as to the scope of use or functionality of the various embodiments described.

Other well-known computer environments, configurations, and/or systems that may be appropriate for use with the embodiments include, but are not limited to, network PCs, mainframe computers, programmable consumer electronics, set top boxes, game consoles, programmable consumer electronics, personal computers, server computers, hand-held or laptop devices, multiprocessor systems, microprocessor-based systems, and/or the like. For instance, part or all the computing system components could be put into use in a cloud computing environment, where resources and/or services are made available for user devices to consume on a selected basis via a computer network.

Further, while certain aspects have been described using a particular combination of hardware and software, it should be recognized that other combinations of hardware and software are also possible. Certain aspects may be implemented only in hardware, or only in software, or using combinations thereof. The various processes described herein may be implemented on the same processor or different processors in any combination.

Where devices, systems, components or modules are described as being configured to perform certain operations or functions, such configuration may be accomplished, for example, by designing electronic circuits to perform the operation, by programming programmable electronic circuits (such as microprocessors) to perform the operation, such as by executing computer instructions or code, or processors or cores programmed to execute code or instructions stored on a non-transitory memory medium, or any combination thereof. Processes may communicate using a variety of techniques including but not limited to conventional techniques for inter-process communications, and different pairs of processes may use different techniques, or the same pair of processes may use different techniques at different times.

Specific details are given in this disclosure to provide a thorough understanding of the aspects. However, aspects may be practiced without these specific details. For example, well-known circuits, processes, algorithms, structures, and techniques have been shown without unnecessary detail in order to avoid obscuring the aspects. This description provides example aspects only, and is not intended to limit the scope, applicability, or configuration of other aspects. Rather, the preceding description of the aspects may provide those skilled in the art with an enabling description for implementing various aspects. Various changes may be made in the function and arrangement of elements.

The specification and drawings are, accordingly, to be regarded in an illustrative rather than a restrictive sense. It may, however, be evident that additions, subtractions, deletions, and other modifications and changes may be made thereunto without departing from the broader spirit and scope as set forth in the claims. Thus, although specific aspects have been described, these are not intended to be limiting. Various modifications and equivalents are within the scope of the following claims.

What is claimed is:

1. A computer-implemented method including:
accessing, for each time point of a set of time points, multimodal data including a retinal scan associated with one or more retinal modalities and non-retinal data associated with a subject;
generating, via one or more machine-learning models, a set of feature vectors associated with the multimodal data over the set of time points;
generating, via the one or more machine-learning models, a temporal feature vector by aggregating the set of feature vectors over the set of time points;
generating, via a prediction model of the one or more machine-learning models, a disease-prediction metric by processing the temporal feature vector, wherein:
the prediction model is configured to predict with respect to a current time point or a future time point whether the subject has or will have one or more diseases based on the multimodal data;
the one or more machine-learning models were trained using a loss function that predicts whether the set of feature vectors representing retinal degradation corresponds to aging or a disease incident; and
outputting a result corresponding to the disease-prediction metric.

2. The computer-implemented method of claim 1, wherein generating each feature vector of the set of feature vectors further including:
generating a first embedding vector associated with a set of image-based modalities via a first machine-learning model of the one or more machine-learning models, wherein the set of image-based modalities comprises the retinal scan from the one or more retinal modalities and medical imaging data from the non-retinal data;
generating a second feature vector associated with a set of text-based modalities via a second machine-learning model of the one or more machine-learning models, wherein the set of text-based modalities comprises textual data from the non-retinal data; and
aggregating the first embedding vector and the second embedding vector to generate a feature vector of the set of feature vectors via a third machine-learning model of the one or more machine-learning models.

3. The computer-implemented method of claim 1, wherein the prediction model includes:
generating predictions from a set of sub-models including a Cox model, an incident model, an aging model and a patient information model; and
aggregating, via an ensemble model of the one or more machine-learning models, the predictions from the set of sub-models to generate the disease-prediction metric, wherein the ensemble model integrates weighted predictions from each sub-model of the set of sub-models.

4. The computer-implemented method of claim 1, wherein the result corresponding to the disease-prediction metric includes a risk score, a probability, or a predicted change representing a likelihood of the one or more diseases.

5. The computer-implemented method of claim 1, wherein the one or more retinal modalities comprise: fundus, optical coherence tomography (OCT), fluorescence angiography (FA), functional magnetic resonance imaging (fMRI), ocular ultrasound imaging, multispectral imaging (MSI), scanning laser ophthalmoscopy (SLO) and optical radiography.

6. The computer-implemented method of claim 1, wherein the non-retinal data comprises one or more of: demographic data, anthropometric data, comorbidities data, medical imaging data, or lifestyle indicators data corresponding to the set of time points.

7. The computer-implemented method of claim 1, wherein the prediction model is configured to predict the one or more diseases including cardiovascular diseases, diabetic retinopathy, age-related macular degeneration, glaucoma, hypertensive retinopathy, stroke, multiple sclerosis, Alzheimer's disease, Parkinson's disease, chronic kidney disease, retinal vein occlusion, macular edema, systemic inflammatory diseases including lupus and rheumatoid arthritis, and ocular or metastatic cancers based on the multimodal data.

8. A system comprising:
one or more data processors; and
a non-transitory computer readable storage medium containing instructions which, when executed on the one or more data processors, cause the one or more data processors to perform operations including:
accessing, for each time point of a set of time points, multimodal data including a retinal scan associated with one or more retinal modalities and non-retinal data associated with a subject;
generating, via one or more machine-learning models, a set of feature vectors associated with the multimodal data over the set of time points;
generating, via the one or more machine-learning models, a temporal feature vector by aggregating the set of feature vectors over the set of time points;
generating, via a prediction model of the one or more machine-learning models, a disease-prediction metric by processing the temporal feature vector, wherein:
the prediction model is configured to predict with respect to a current time point or a future time point whether the subject has or will have one or more diseases based on the multimodal data;
the one or more machine-learning models were trained using a loss function that predicts whether the set of feature vectors representing retinal degradation corresponds to aging or a disease incident; and
outputting a result corresponding to the disease-prediction metric.

9. The system of claim 8, wherein generating each feature vector of the set of feature vectors further including:
generating a first embedding vector associated with a set of image-based modalities via a first machine-learning model of the one or more machine-learning models, wherein the set of image-based modalities comprises the retinal scan associated with the one or more retinal modalities and the non-retinal data;
generating a second feature vector associated with a text-based modalities via a second machine-learning model of the one or more machine-learning models, wherein the set of text-based modalities comprises the non-retinal data; and
aggregating the first embedding vector and the second embedding vector to generate a feature vector of the set of feature vectors via a third machine-learning model of the one or more machine-learning models.

10. The system of claim 8, wherein the prediction model includes:
generating predictions from a set of sub-models including a Cox model, an incident model, an aging model and a patient information model; and
aggregating, via an ensemble model of the one or more machine-learning models, the predictions from the set of sub-models to generate the disease-prediction metric, wherein the ensemble model integrates weighted predictions from each sub-model of the set of sub-models.

11. The system of claim 8, wherein the result corresponding to the disease-prediction metric includes a risk score, a probability, or a predicted change representing a likelihood of the one or more diseases.

12. The system of claim 8, wherein the one or more retinal modalities comprise: fundus, optical coherence tomography (OCT), fluorescence angiography (FA), functional magnetic resonance imaging (fMRI), ultrasound imaging, multispectral imaging (MSI), scanning laser ophthalmoscopy (SLO) and optical radiography.

13. The system of claim 8, wherein the non-retinal data comprises one or more of: demographic data, anthropometric data, comorbidities data, medical imaging data, or lifestyle indicators data corresponding to the set of time points.

14. The system of claim 8, wherein the prediction model is configured to predict the one or more diseases including cardiovascular diseases, diabetic retinopathy, age-related macular degeneration, glaucoma, hypertensive retinopathy, stroke, multiple sclerosis, Alzheimer's disease, Parkinson's disease, chronic kidney disease, retinal vein occlusion, macular edema, systemic inflammatory diseases including lupus and rheumatoid arthritis, and ocular or metastatic cancers based on the multimodal data.

15. A computer-program product tangibly embodied in a non-transitory machine-readable storage medium, including instructions configured to cause one or more data processors to perform operations including:
accessing, for each time point of a set of time points, multimodal data including a retinal scan associated with one or more retinal modalities and non-retinal data associated with a subject;
generating, via one or more machine-learning models, a set of feature vectors associated with the multimodal data over the set of time points;
generating, via the one or more machine-learning models, a temporal feature vector by aggregating the set of feature vectors over the set of time points;
generating, via a prediction model of the one or more machine-learning models, a disease-prediction metric by processing the temporal feature vector, wherein:
the prediction model is configured to predict with respect to a current time point or a future time point whether the subject has or will have one or more diseases based on the multimodal data;
the one or more machine-learning models were trained using a loss function that predicts whether the set of feature vectors representing retinal degradation corresponds to aging or a disease incident; and
outputting a result corresponding to the disease-prediction metric.

16. The computer-program product of claim 15, wherein generating each feature vector of the set of feature vectors further including:
generating a first embedding vector associated with a set of image-based modalities via a first machine-learning model of the one or more machine-learning models, wherein the set of image-based modalities comprises the retinal scan associated with the one or more retinal modalities and the non-retinal data;
generating a second feature vector associated with a text-based modalities via a second machine-learning model of the one or more machine-learning models, wherein the set of text-based modalities comprises the non-retinal data; and
aggregating the first embedding vector and the second embedding vector to generate a feature vector of the set of feature vectors via a third machine-learning model of the one or more machine-learning models.

17. The computer-program product of claim 15, wherein the prediction model includes:
generating predictions from a set of sub-models including a Cox model, an incident model, an aging model or a patient information model; and
aggregating, via an ensemble model of the one or more machine-learning models, the predictions from the set of sub-models to generate the disease-prediction metric, wherein the ensemble model integrates weighted predictions from each sub-model of the set of sub-models.

18. The computer-program product of claim 15, wherein the result corresponding to the disease-prediction metric includes a risk score, a probability, or a predicted change representing a likelihood of the one or more diseases.

19. The computer-program product of claim 15, wherein the one or more retinal modalities comprise: fundus, optical coherence tomography (OCT), fluorescence angiography (FA), functional magnetic resonance imaging (fMRI), ultrasound imaging, multispectral imaging (MSI), scanning laser ophthalmoscopy (SLO) and optical radiography.

20. The computer-program product of claim 15, wherein the non-retinal data comprises one or more of: demographic data, anthropometric data, comorbidities data, medical imaging data, or lifestyle indicators data corresponding to the set of time points.
